(19) United States
(12) Patent Application Publication
(10) Pub. No.: US 2017/0071771 A1
(43) Pub. Date: Mar. 16, 2017

(54) SELF-EXPANDABLE STENT DELIVERY SYSTEM

(71) Applicant: TERUMO KABUSHIKI KAISHA, Tokyo (JP)

(72) Inventor: Kinya HARADA, Fuji-city (JP)

(73) Assignee: TERUMO KABUSHIKI KAISHA, Tokyo (JP)

(21) Appl. No.: 15/259,698

(22) Filed: Sep. 8, 2016

(30) Foreign Application Priority Data
Sep. 10, 2015 (JP) 2015-178891

(51) Int. Cl. A61F 2/966; A61F 2/844

(52) U.S. Cl. CPC A61F 2/966; A61F 2/844; A61F 2250/0098

(57) ABSTRACT

A stent delivery system includes an inner tube having a guide wire lumen formed therein, through which a guide wire is inserted, an outer tube including a lumen, through which the inner tube is inserted, and arranged to be relatively movable with respect to the inner tube, a distal member fixed to a distal end of the inner tube, and a stent arranged between a distal portion of the inner tube and a distal portion of the outer tube and configured to expand and deform after being released from between the inner tube and the outer tube along with movement of the outer tube. With the stent released, the inner tube and the distal member are configured to be removable from the outer tube via the lumen of the outer tube.

### CROSS-REFERENCES TO RELATED APPLICATIONS

This application claims priority to Japanese Patent Application No. 2015-178891 filed on Sep. 10, 2015, the entire content of which is incorporated herein by reference.

### TECHNICAL FIELD

The disclosure herein relates to a self-expandable stent delivery system.

### BACKGROUND DISCUSSION

A stent delivery system is widely known as a medical device used to deliver a stent having self-expandability to the desired portion of a living body lumen or the like.

For example, a stent delivery system disclosed in International Publication No. WO2007/122901 includes an inner tube having a lumen formed therein, into which a guide wire is inserted through, and an outer tube arranged to cover a distal portion of the inner tube. The stent delivery system is configured such that inserting the guide wire through the guide wire lumen of the inner tube enables insertion of the inner tube and the outer tube up to the desired portion in the living body along the guide wire. After the stent is released into the living body and is then indwelled therein, the outer tube and the inner tube are removed out of the living body.

In treating a lesion (a stenosed site or the like) formed in a blood vessel or the like, various procedures may be performed after the stent is indwelled as to post-dilate the lesion in which the stent is indwelled, using a balloon catheter, and to indwell the stent in another lesion. For example, in treating the lower extremity, after an above-knee portion, such as the superficial femoral artery, is treated using a stent delivery system, a below-knee portion, such as the popliteal artery or the tibial artery, may be treated using another medical device. In such a procedure, if, after treatment for the superficial femoral artery is finished, the stent delivery system is removed and another medical device, in place of the stent delivery system, must then be inserted into the popliteal artery or the tibial artery, not only is a prompt procedure hindered but also a burden on the patient may increase.

The disclosure herein is directed to solving the above-mentioned problems, and provides a self-expandable stent delivery system enabling a prompt and minimally invasive procedure and which is capable of contributing to medical economy.

### SUMMARY

A self-expandable stent delivery system according to the disclosure includes an inner tube having a guide wire lumen formed therein through which a guide wire is inserted, an outer tube including a lumen through which the inner tube is inserted and arranged to be relatively movable with respect to the inner tube, a distal member fixed to a distal end of the inner tube, and a self-expandable stent arranged between a distal portion of the inner tube and a distal portion of the outer tube and configured to expand and deform after being released from between the inner tube and the outer tube along with movement of the outer tube. With the self-expandable stent released, the inner tube and the distal member are configured to be removable from the outer tube via the lumen of the outer tube.

According to the self-expandable stent delivery system configured as described above, since the inner tube and the distal member can be removed from the outer tube after the stent is indwelled in the living body lumen, another medical device can be inserted into the living body lumen via the lumen of the outer tube. In this way, since utilizing the outer tube as a guiding catheter for a medical device can contribute to medical economy and can eliminate the necessity of inserting a guiding catheter, a prompt procedure can be performed, so that a minimally invasive procedure with a reduced burden on the patient can be implemented.

### BRIEF DESCRIPTION OF THE DRAWINGS

FIG. 1 is a diagram illustrating an overall structure of a self-expandable stent delivery system according to an exemplary embodiment of the disclosure.

FIG. 2 is a partial cross-sectional view of the self-expandable stent delivery system according to the exemplary embodiment.

FIG. 3 is a partial cross-sectional view of the self-expandable stent delivery system with a distal member and an inner tube removed.

FIGS. 4(A) and 4(B) are diagrams illustrating a self-expandable stent, which is mounted in the self-expandable stent delivery system according to the exemplary embodiment.

FIG. 5 is a flowchart illustrating a method of treatment using the self-expandable stent delivery system according to the exemplary embodiment.

FIGS. 6(A) and 6(B) are diagrams illustrating the method of treatment using the self-expandable stent delivery system according to the exemplary embodiment.

FIGS. 7(A) and 7(B) are diagrams illustrating the method of treatment using the self-expandable stent delivery system according to the exemplary embodiment.

FIGS. 8(A) and 8(B) are diagrams illustrating the method of treatment using the self-expandable stent delivery system according to the exemplary embodiment.

FIGS. 9(A) and 9(B) are diagrams illustrating the method of treatment using the self-expandable stent delivery system according to the exemplary embodiment.

### DETAILED DESCRIPTION

Hereinafter, exemplary embodiments of the disclosure herein will be described with reference to the accompanying drawings. Furthermore, the following description should not be construed to limit the technical scope and the meaning of each term set forth in the claims. Moreover, dimensional ratios illustrated in the drawings are exaggerated for the purpose of illustration and may be different from the actual ratios.

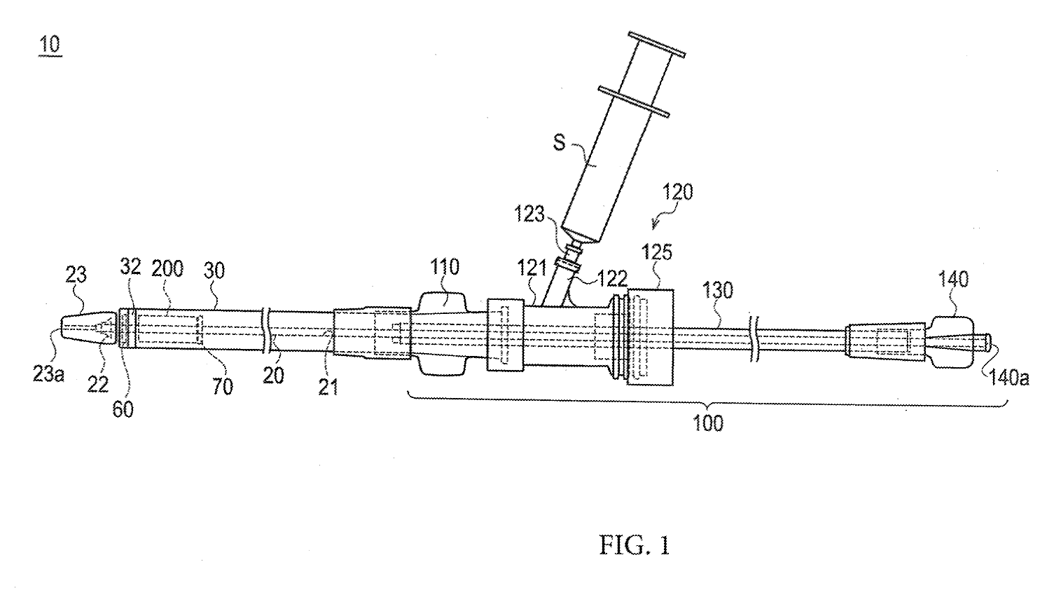

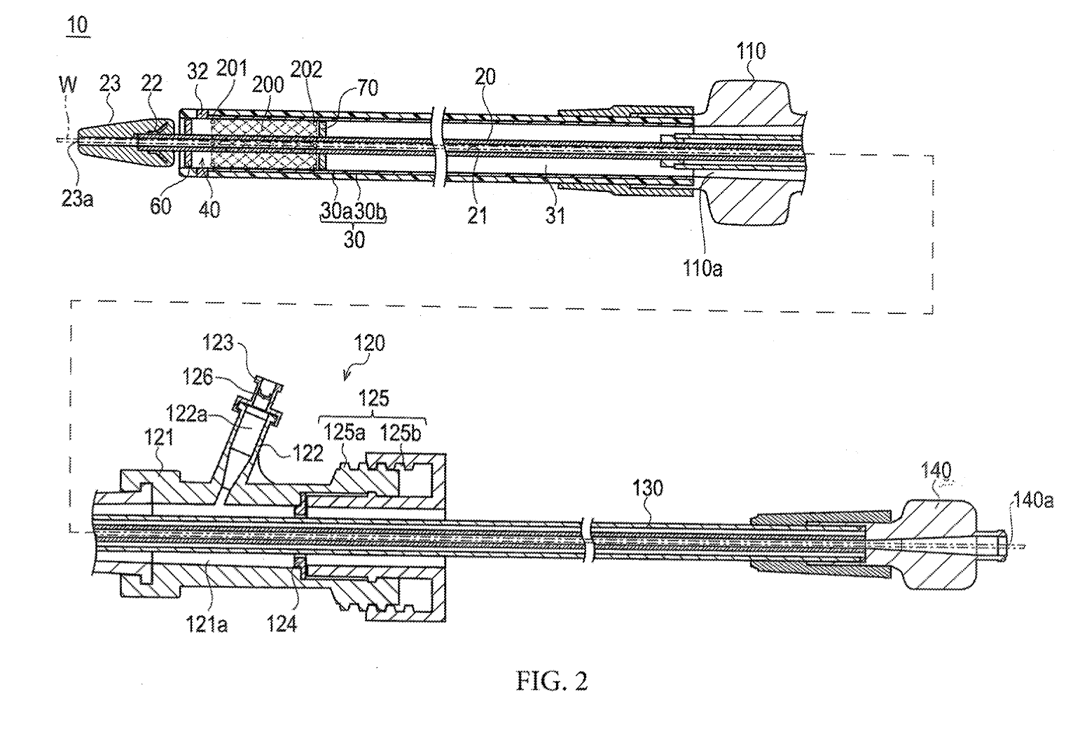

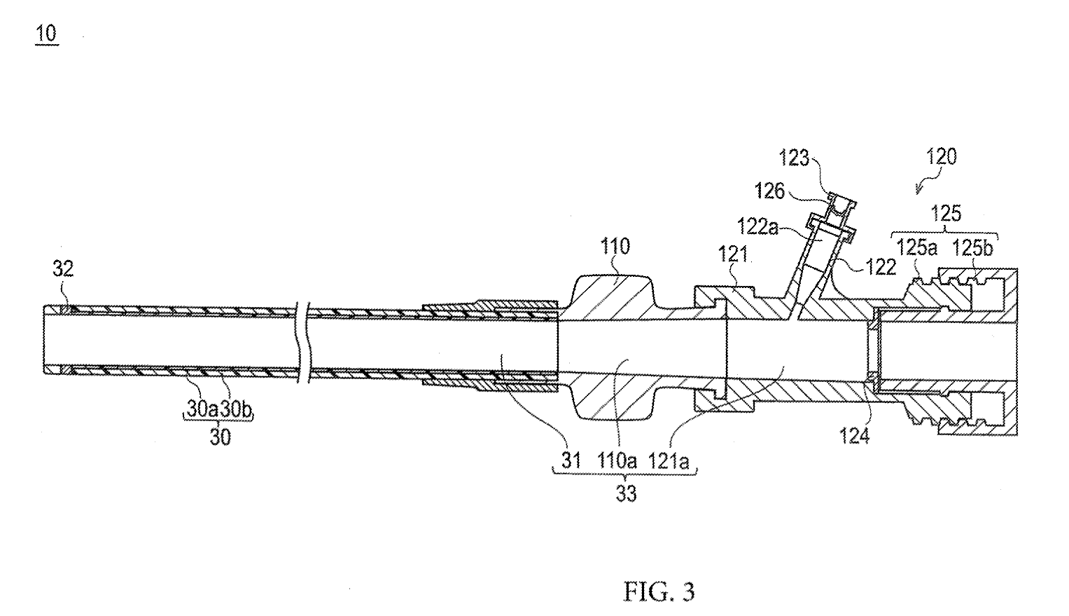

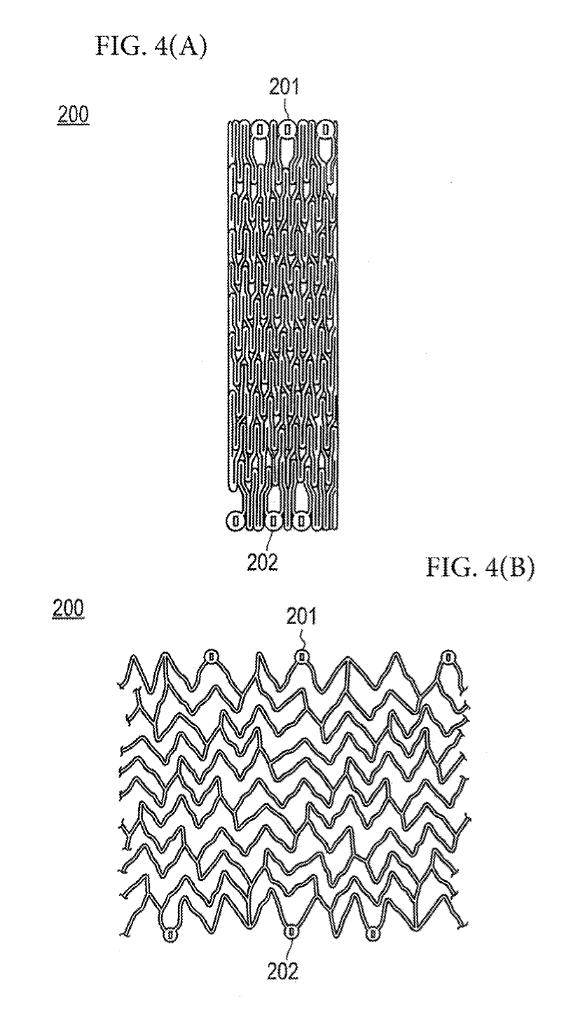

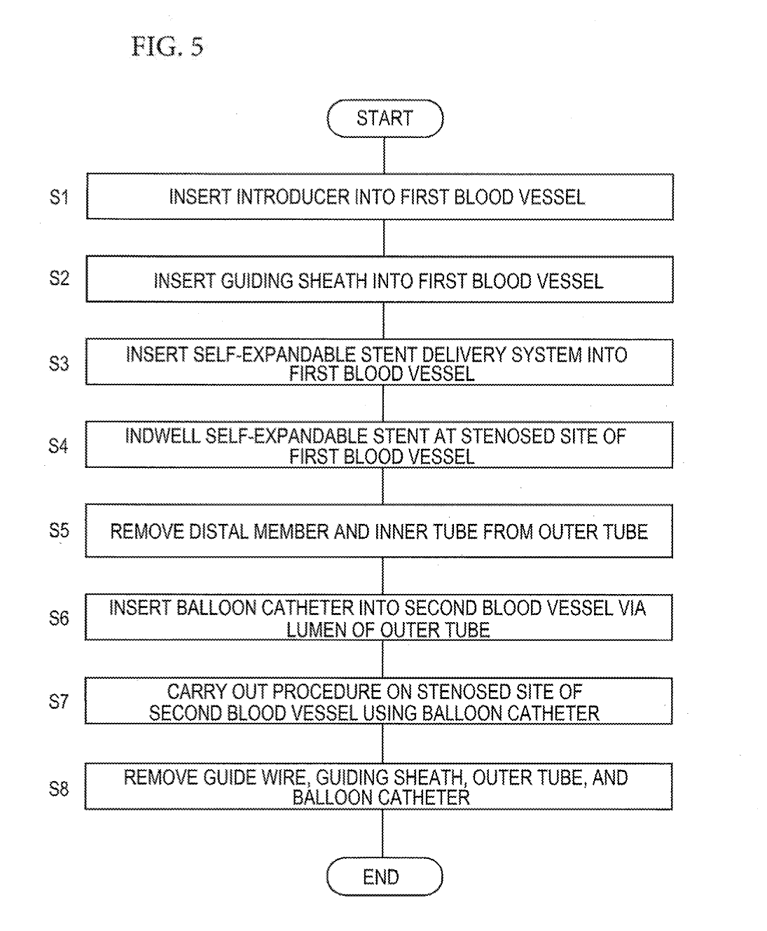

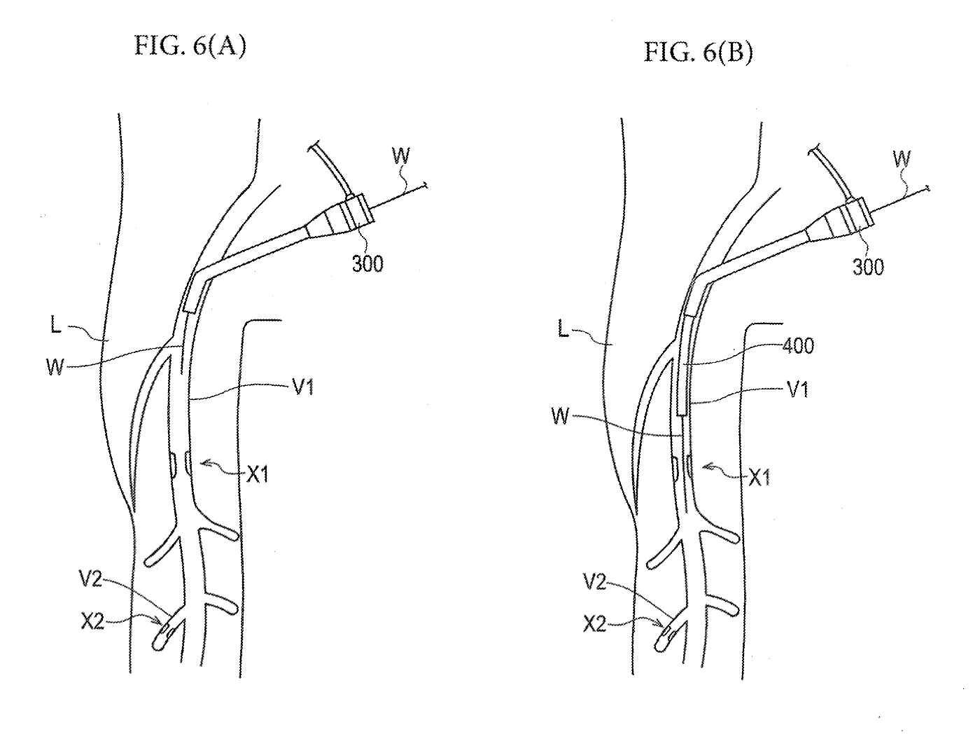

FIG. 1 and FIG. 2 are diagrams illustrating an overall structure of a self-expandable stent delivery system (hereinafter, referred to as a “stent delivery system”) according to an exemplary embodiment. FIG. 3 is a diagram illustrating an overall structure of the stent delivery system with a distal member and an inner tube removed. FIGS. 4(A) and 4(B) are diagrams illustrating a self-expandable stent (hereinafter referred to as a “stent”), which is mounted in the stent delivery system. FIG. 5 is a flowchart illustrating a method of treatment using the stent delivery system. FIG. 6(A) to FIG. 9(B) are diagrams illustrating the method of treatment using the stent delivery system.

As illustrated in FIG. 1 and FIG. 2, a stent delivery system 10 according to an exemplary embodiment of the disclosure herein includes, generally stated, an inner tube 20 having a guide wire lumen 21 formed therein, through which a guide wire W is inserted, an outer tube 30 arranged to cover a distal portion side of the inner tube 20, a stent 200 arranged between a distal portion of the inner tube 20 and a distal portion of the outer tube 30 and configured to expand and deform after being released from between the inner tube 20 and the outer tube 30 along with movement of the outer tube 30, and a hand operation portion 100 arranged at a proximal end side of the inner tube 20 and configured to be grippable.

In the disclosure herein, the side at which the insertion into the living body is carried out is referred to as a “distal end” or a “distal end side”, and the side at which the hand operation portion 100 is located is referred to as a “proximal end” or a “proximal end side”.

As illustrated in FIG. 2, the inner tube 20 is formed of an elongated tubular body in which the guide wire lumen 21 extending from the distal end to the proximal end is formed. The guide wire W, which inserts the stent delivery system 10 to the lesion in the living body lumen, is inserted through the guide wire lumen 21. The guide wire W to be used can be, for example, a known one made from a metallic material, such as a stainless steel or nitinol.

The stent delivery system 10 in the exemplary embodiment is configured as an over-the-wire (OTW) type, in which the guide wire lumen 21 continuously communicates from a distal end opening portion 23a provided at the distal end to a port 140a provided at the proximal end of the hand operation portion 100.

As illustrated in FIG. 2, a distal member 23 is arranged at the most distal end of the stent delivery system 10. The distal member 23 is fixed to the distal portion of the inner tube 20 via a predetermined fastener 22.

The distal member 23 is formed in such a tapered shape that the diameter thereof gradually decreases toward the distal end, in consideration of the insertability into the living body lumen. The distal end opening portion 23a, through which the guide wire W is inserted, is formed at the distal end of the distal member 23. The distal member 23, for example, can be formed by a member different from the inner tube 20, or it can also be integrally formed with the same member as the inner tube 20. The material used to form the distal member 23 is desirably a material having flexibility, and can be, for example, a known resin material.

The fastener 22 is embedded in the distal member 23. The fastener 22 functions to prevent detachment of the distal member 23. The fastener 22 can be formed from, for example, a metallic material such as a stainless steel.

As illustrated in FIG. 2, the proximal end of the inner tube 20 is inserted through the port 140a, at which the guide wire W is taken in and out. The proximal end of the inner tube 20 can be, for example, fixed to the port 140a with adhesive or the like.

The material used to form the inner tube 20 is desirably a material having flexibility, and can be, for example, polyolefin such as polyethylene or polypropylene, polyamide, polyester such as polyethylene terephthalate, fluorine-based polymer such as ETFE, PEEK, or polyimide.

The outer tube 30 is formed of an elongated tubular body, and is equipped with a lumen 31, through which the inner tube 20 is inserted. Furthermore, as illustrated in FIG. 2, the outer tube 30 is arranged at the outer surface side of the inner tube 20 in such a way as to be relatively movable with respect to the inner tube 20.

With the inner tube 20 and the outer tube 30 assembled, the distal portion of the inner tube 20 is arranged to protrude beyond the distal portion of the outer tube 30. A gap portion 40, which is provided to accommodate the stent 200, is formed between the distal portion of the inner tube 20 and the distal portion of the outer tube 30.

The gap portion 40 is formed by a space partitioned between a distal marker 60 and a stent stopper 70, which are arranged inside the outer tube 30, and the inner wall of the outer tube 30. The stent 200, in the state of not yet being indwelled in the lesion in the living body lumen, is accommodated in the gap portion 40 while being compressed in the radially inward direction as illustrated in FIG. 4(A).

As illustrated in FIG. 2, the outer tube 30 includes an inner layer 30a and an outer layer 30b which covers the outer surface of the inner layer 30a. Further, a reinforcement body serving to improve the kink resistance of the outer tube 30 can be arranged between the inner layer 30a and the outer layer 30b.

The material used to form the inner layer 30a can be, for example, fluorine-based resin such as polytetrafluoroethylene (PTFE), or polyethylene. Furthermore, the material used to form the outer layer 30b can be, for example, polyamide resin, or polyester resin such as polyethylene terephthalate (PET). The material used to form the reinforcement body can be, for example, metallic wires braided in a net shape.

A ring-shaped marker 32 having radiopacity is arranged at the distal portion of the outer tube 30. The marker 32 can be arranged between the inner layer 30a and the outer layer 30b. The material used to form the marker 32 is not specifically limited as long as it is a material having radiopacity, and can be, for example, metal such as platinum, gold, silver, iridium, titanium, or tungsten, or an alloy of some of those metals.

The outer tube 30 can be formed, for example, with an outer diameter of 0.5 mm to 4.0 mm, and, in the case of being used for a relatively thin lumen such as the peripheral blood vessel of the lower extremity, is desirably formed with an outer diameter of 0.8 mm to 2.0 mm. The lumen 31 of the outer tube 30 can be formed, for example, with an inner diameter of 0.2 mm to 1.8 mm.

The inner tube 20 is formed with an outer diameter smaller than the inner diameter of the lumen 31 of the outer tube 30, and can be formed, for example, with an outer diameter of 0.50 mm to 1.50 mm.

The distal member 23 is formed with a maximum outer diameter smaller than the inner diameter of the lumen 31 of the outer tube 30, and can be formed, for example, with a maximum outer diameter of 0.18 mm to 1.78 mm.

Since the outer diameter of each of the inner tube 20 and the distal member 23 is formed smaller than the inner diameter of the lumen 31 of the outer tube 30, as mentioned above, the inner tube 20 and the distal member 23 are able to be removed from the outer tube 30 via the lumen 31 of the outer tube 30.

The stent 200, in the state of being accommodated in the gap portion 40, receives constraint force from the inner surface of the outer tube 30, and is thus restricted from expanding and deforming in the radially outward direction. FIG. 4(A) illustrates an example of a state in which the stent 200 is contracted.

When the outer tube 30 moves toward the proximal end side with respect to the inner tube 20 and the gap portion 40 becomes exposed to the outside, the stent 200 is released from the constraint imposed by the inner surface of the outer tube 30 and, thus, expands and deforms in the radially outward direction as illustrated in FIG. 4(B).

The stent 200 can be formed, for example, with an outer diameter in an expanded state of 2 mm to 12 mm. Furthermore, the stent 200 can be formed, for example, with a wall thickness of 0.05 mm to 0.25 mm.

The stent 200 to be used can be, as appropriate, a known stent having self-expandability. For example, a stent formed of a superelastic alloy such as a nickel-titanium alloy, or a stent formed of a polymer material or another metallic material, can be used. Examples of the polymer material include polyolefin such as polyethylene or polypropylene, polyester such as polyethylene terephthalate, and fluorine-containing polymer such as polytetrafluoroethylene or tetrafluoroethylene-ethylene copolymer. Examples of the metallic material include cobalt-chrome alloy, stainless steel, iron, titanium, aluminum, tin, and zinc-tungsten alloy.

As illustrated in FIGS. 4(A) and 4(B), the stent 200 includes a distal side stent marker 201 and a proximal side stent marker 202, which have radiopacity.

The distal side stent marker 201 and the proximal side stent marker 202 can be formed of, for example, a material having radiopacity. The material having radiopacity to be used can be the same material as that of the marker 32, which is arranged at the outer tube 30.

As illustrated in FIG. 2, the distal marker 60, which is arranged inside the outer tube 30, is fixed to the outer surface of the inner tube 20. Since the outer diameter of the distal marker 60 is formed substantially equal to the inner diameter of the outer tube 30, frictional force acts between the outer surface of the inner tube 20 and the inner surface of the outer tube 30 via the distal marker 60. The frictional force thus prevents the outer tube 30 from unexpectedly moving with respect to the inner tube 20. Furthermore, the distal marker 60 can be formed of a material having radiopacity as with the distal side stent marker 201 and the proximal side stent marker 202.

The stent stopper 70 is arranged near the proximal end side of the stent 200 when the stent is accommodated in the gap portion 40. When an operation is performed to move the outer tube 30 toward the proximal end side with respect to the inner tube 20, the proximal end of the stent 200 comes into contact with the stent stopper 70. The stent 200 is restricted by such contact from moving toward the proximal end side. Since the outer tube 30 further moves toward the proximal end side independent from the stent 200, the stent 200 with the proximal end thereof supported by the stent stopper 70 is expelled from between the inner tube 20 and the outer tube 30 and is then released to a predetermined indwelling site (lesion).

As illustrated in FIG. 1, the stent delivery system 10 includes the hand operation portion 100, which is arranged at the proximal end side of the outer tube 30.

The hand operation portion 100 includes an outer tube hub 110, to which the proximal portion of the outer tube 30 is attached, a connector portion (Y connector portion) 120, which is connected to the proximal end of the outer tube hub 110 and is provided to be movable together with the outer tube 30, a proximal shaft 130, which covers the inner tube 20 at the proximal end side of the connector portion 120, and an inner tube hub 140, to which the proximal end of the inner tube 20 is attached.

As illustrated in FIG. 2, the outer tube hub 110 is connected to the proximal portion of the outer tube 30 in a liquid-tight manner. A lumen 110a of the outer tube hub 110 communicates with the lumen 31 of the outer tube 30. The outer tube hub 110 can be formed of, for example, a known resin material or metallic material.

The connector portion 120 includes a main tube 121, which communicates with the lumen 31 of the outer tube 30 and through which the inner tube 20 is movably inserted, a branch tube 122, which branches from the main tube 121, a connecting tube 123, which is provided between the branch tube 122 and a fluid supply source S (referring to FIG. 1), an on-off valve 124, which is arranged to freely open and close a lumen 121a of the main tube 121, and a cap portion 125, which is provided at the proximal portion of the main tube 121. The lumen 121a of the main tube 121 communicates with the lumen 31 included in the outer tube 30 via the lumen 110a of the outer tube hub 110.

The main tube 121 of the connector portion 120 is arranged to be relatively movable with respect to the inner tube 20. As the main tube 121 of the connector portion 120 is moved toward the proximal end side, the outer tube 30, which is connected to the main tube 121 via the outer tube hub 110, is also moved toward the proximal end side.

The branch tube 122 has a port 122a formed therein, which communicates with the lumen 31 of the outer tube 30 via the main tube 121. As illustrated in FIG. 1, a known fluid supply source S, such as a syringe, is coupled to the branch tube 122 via the connecting tube 123. For example, a fluid, such as physiological salt solution, contrast agent, or Ringer's solution, can be supplied to the lumen 31 of the outer tube 30 via a tube and the branch tube 122.

The connecting tube 123 connects the branch tube 122 and the fluid supply source S to each other. A check valve (corresponding to a valve body) 126 is provided in the lumen of the connecting tube 123. The check valve 126 allows a fluid supplied from the fluid supply source S to flow to the lumen 31 of the outer tube 30 but, on the other hand, prevents a fluid from flowing from the lumen 31 of the outer tube 30 to the port 122a. The check valve 126 is formed of a material having flexibility, and can be formed of, for example, a known elastic material, such as natural rubber, synthetic rubber, or silicone rubber.

The on-off valve 124 is arranged to surround the outer periphery of the proximal shaft 130. In response to an opening or closing operation performed, the on-off valve 124 opens or closes a clearance formed between the on-off valve 124 and the proximal shaft 130. The opening or closing operation on the on-off valve 124 can be performed using the cap portion 125. The on-off valve 124 can be formed of the same material as that of the check valve 126.

The cap portion 125 includes a male thread portion 125a, which is formed on the outer surface of the proximal portion of the main tube 121, and a female thread portion 125b, which is threadedly engaged with the male thread portion 125a. When the cap portion 125 is rotated with the female thread portion 125b engaged with the male thread portion 125a, the on-off valve 124, which is arranged at the distal end side of the cap portion 125, is pressed and is thus compressed in the radially inward direction. When tightening of the cap portion 125 is loosened, the compressed state of the on-off valve 124 is canceled.

When the on-off valve 124 is in an open state, the connector portion 120 is relatively movable with respect to the proximal shaft 130. In other words, the outer tube 30, which is connected to the connector portion 120, is relatively movable with respect to the inner tube 20, which is inserted thorough the proximal portion shaft 130.

When the on-off valve 124 is in a closed state, the on-off valve 124 is in pressed contact with the outer peripheral surface of the proximal shaft 130. In this state, a liquid-tight state is maintained at a portion nearer the distal end side than the on-off valve 124 (that is, a portion distal to the on-off valve 124). Furthermore, since the movement of the connector portion 120 relative to the proximal shaft 130 is restricted by pressed-contact force exerted by the on-off valve 124, the movement of the outer tube 30 relative to the inner tube 20 is also restricted.

Bringing the on-off valve 124 into pressed contact with the outer peripheral surface of the proximal shaft 130 enables supplying a fluid, such as physiological salt solution, to the lumen 31 of the outer tube 30 via the branch tube 122. Furthermore, since, in inserting the stent delivery system 10 into the living body lumen, any deviation in relative position between the outer tube 30 and the inner tube 20 can be prevented, operability can be improved.

Each portion (the main tube 121, the branch tube 122, and the cap portion 125) of the connector portion 120 can be formed of, for example, a known resin material or metallic material.

The proximal shaft 130 has a hollow pipe-like shape through which the inner tube 20 is insertable. The proximal portion shaft 130 can be formed of, for example, stainless steel or nitinol.

The proximal end of the proximal shaft 130 is fixed to, for example, the inner tube hub 140 included in the hand operation portion 100, as illustrated in FIG. 2. The inner tube 20, as well as the proximal shaft 130, are inserted into the inner tube hub 140. Furthermore, the inner tube 20 is connected to the port 140a, which is arranged at the proximal end of the inner tube hub 140. The port 140a has a communicating hole formed therein, which communicates with the guide wire lumen 21 included in the inner tube 20. In performing a procedure using the stent delivery system 10, the guide wire W can be introduced via the distal end opening portion 23a of the distal member 23 arranged at the distal end and, then, the guide wire W can be caused to protrude beyond the proximal end of the port 140a.

The inner tube hub 140 is connected to the proximal ends of the proximal shaft 130 and the inner tube 20. The inner tube hub 140 has the port 140a formed therein. The port 140a is configured such that, for example, a fluid tube (not illustrated) coupled to a fluid supply source (not illustrated), which supplies a fluid such as physiological salt solution, contrast agent, or Ringer's solution, is allowed to be connected to the port 140a in a liquid-tight and air-tight manner, as with the branch tube 122. Examples of the material used to form the inner tube hub 140 include thermoplastic resin, such as polycarbonate, polyamide, polysulfone, polyarylate, and methacrylate-styrene copolymer.

Next, a method of treatment using the stent delivery system 10 according to the exemplary embodiment is described with reference to FIG. 5. According to the stent delivery system 10 in the exemplary embodiment, after the treatment of a lesion is carried out, the outer tube 30 can be used as a guiding catheter for introducing another medical device 500. Therefore, after the stent 200 is indwelled, the medical device 500 can be used to perform various procedures on a lesion in which the stent 200 is indwelled, or on another lesion.

In the following description, the treatment of a stenosed site (lesion area), as a treatment site, formed by thrombus or the like dwelling in a blood vessel of the lower extremity (right leg) L of a patient is described. As therapeutic objectives, a first stenosed site X1 formed in the superficial femoral artery located at the thigh portion of the lower extremity L of the patient and a second stenosed site X2 formed in a peripheral blood vessel leading to the popliteal artery located below the knee, as illustrated in FIG. 6(A), are described by way of example. After the treatment of the first stenosed site (first lesion) X1 is carried out using the stent delivery system 10, the treatment of the second stenosed site (second lesion) X2, which is another therapeutic objective, is carried out using the medical device 500, which is different from the stent delivery system 10. An example in which a balloon catheter is used as the medical device 500 is described. Hereinafter, the superficial femoral artery is referred to as a “first blood vessel V1”, and the peripheral blood vessel leading to the popliteal artery is referred to as a “second blood vessel V2”.

First, in step S1, an introducer 300 is inserted into the first blood vessel V1 as illustrated in FIG. 6(A). More specifically, first, the first blood vessel V1 is punctured with a puncture needle (not illustrated) from the skin of the lower extremity L. Next, the guide wire W is inserted into the first blood vessel V1 via the lumen of the puncture needle. Next, the puncture needle is removed from the first blood vessel V1 while the guide wire W remains indwelled in the first blood vessel V1. After that, the introducer 300 is inserted into the first blood vessel V1 along the guide wire W.

Next, in step S2, a guiding sheath 400 is inserted into the first blood vessel V1 via the introducer 300, as illustrated in FIG. 6(B). At this time, it is desirable that the distal portion of the guiding sheath 400 be fixed in the vicinity of the first stenosed site X1 and in the state of being away from the first stenosed site X1.

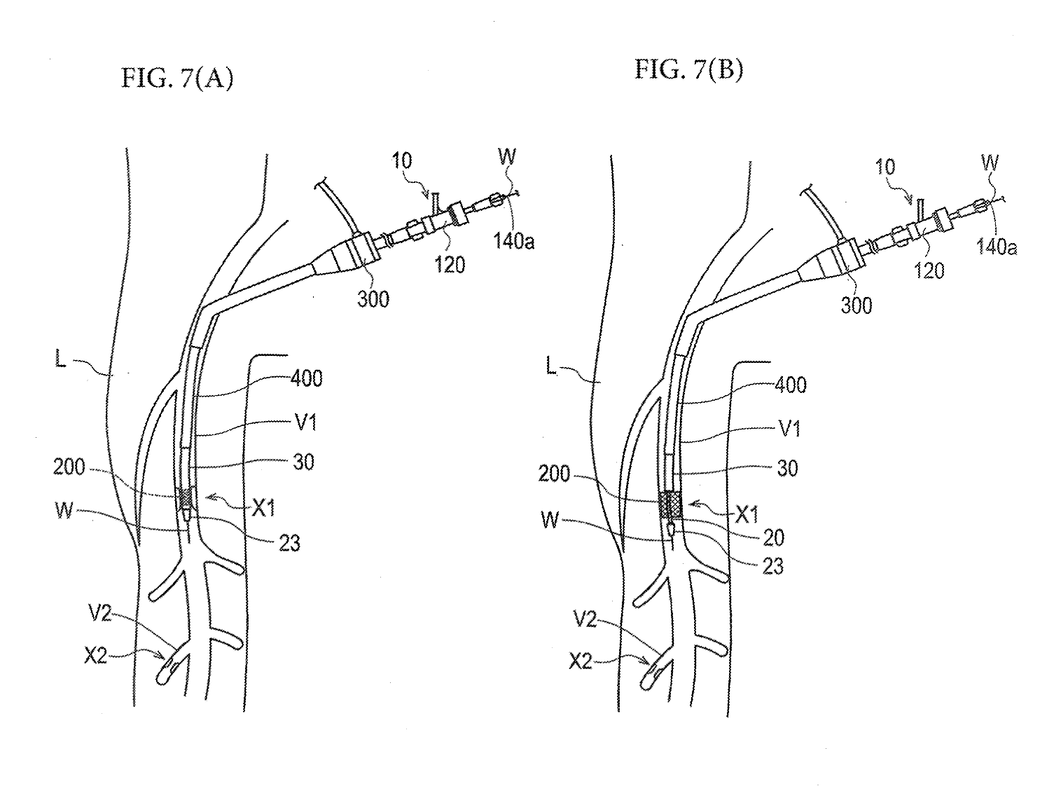

Next, in step S3, the stent delivery system 10 is inserted into the first blood vessel V1 via the guiding sheath 400, as illustrated in FIG. 7(A). More specifically, first, the proximal portion of the guide wire W is inserted into the guide wire lumen 21 of the inner tube 20 via the distal end opening portion 23a of the stent delivery system 10, and then, the guide wire W is led out of the port 140a included in the inner tube hub 140. Moreover, the guide wire W can be replaced by, for example, a penetration guide wire used for penetrating a stenosed site. Next, the stent delivery system 10 is pushed forward in the first blood vessel V1 along the guide wire W, and is then positioned at the first stenosed site X1, which is an indwelling site.

Next, in step S4, the stent 200 is indwelled at the first stenosed site X1 in the first blood vessel V1, as illustrated in FIG. 7(B). More specifically, first, the outer tube 30 is moved toward the proximal end side with respect to the inner tube 20. As the outer tube 30 is moved toward the proximal end side with respect to the inner tube 20, the proximal end of the stent 200 comes into contact with the stent stopper 70, so that the stent 200 is expelled and released from the distal end of the outer tube 30. Since, when the stent is released from the distal end of the outer tube 30, the constraint imposed by the inner surface of the outer tube 30 is completely released, the stent 200 expands returning to its original, uncompressed shape as illustrated in FIG. 4(B). The expanded stent 200 is fixed in close contact with the inner wall of the first stenosed site X1, thus maintaining the lumen shape. With this, the procedure on the first stenosed site X1 using the stent delivery system 10 is completed.

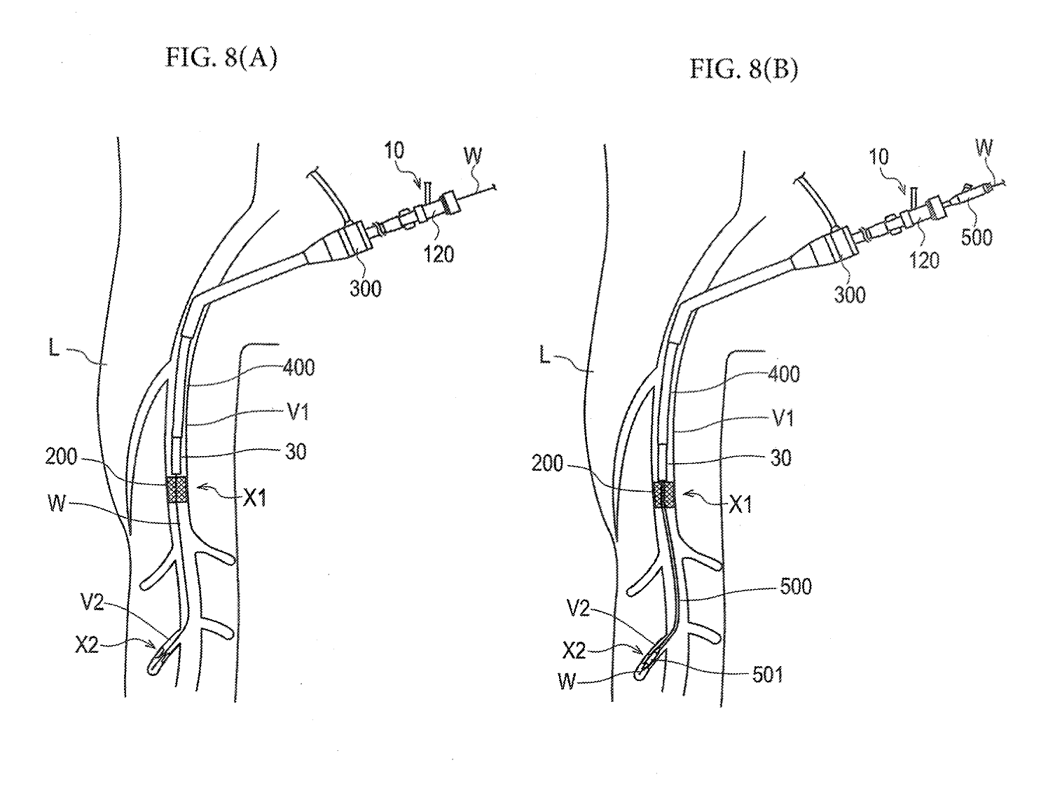

Next, in step S5, as illustrated in FIG. 8(A), while the outer tube 30 is indwelled in the first blood vessel V1, the distal member 23 and the inner tube 20 are removed from the outer tube 30 via the lumen 31 of the outer tube 30. In the stent delivery system 10 with the distal member 23 and the inner tube 20 removed, as illustrated in FIG. 3, the lumen 31 included in the outer tube 30, the lumen 110a included in the outer tube hub 110, and the lumen 121a of the main tube 121 included in the connector portion 120 communicate with one another, thus forming a single lumen 33. The stent delivery system 10 is configured to allow the balloon catheter 500 to be inserted through the thus-formed lumen 33.

Next, in step S6, the balloon catheter 500 is inserted into the second blood vessel V2 via the lumen 31 of the outer tube 30, as illustrated in FIG. 8(B). More specifically, first, the guide wire W is inserted through the second blood vessel V2 up to the second stenosed site X2 (referring to FIG. 8(A)). Next, the balloon catheter 500 is moved forward along the lumen 31 of the outer tube 30, and is then caused to protrude beyond the distal end of the outer tube 30. At this time, while the position of the marker 32 located at the distal portion of the outer tube 30 is confirmed based on a radiographic image captured by X-ray photography, the position of the balloon catheter 500 is adjusted. Next, the balloon catheter 500 is further pushed forward along the guide wire W, and then, a balloon 501 included in the balloon catheter 500 is positioned at the second stenosed site X2 of the second blood vessel V2, which is a target site. In this way, the outer tube 30 of the stent delivery system 10 functions as a guiding catheter that guides the balloon catheter 500 into the second blood vessel V2.

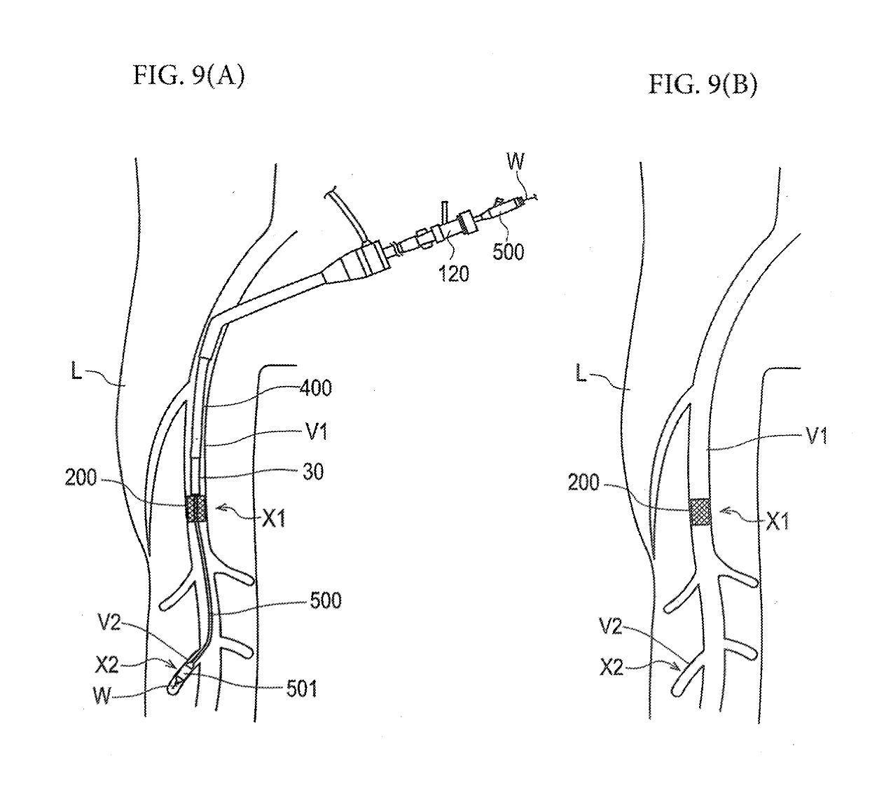

Next, in step S7, as illustrated in FIG. 9(A), the balloon 501 is inflated to dilate the second stenosed site X2 of the second blood vessel V2, thus maintaining the lumen shape. With this, the procedure on the second stenosed site X2 using the balloon catheter 500 is completed.

Finally, in step S8, as illustrated in FIG. 9(B), the guide wire W, the guiding sheath 400, the outer tube 30, and the balloon catheter 500 are removed, so that the treatment is completed.

In this way, the method of treatment using the stent delivery system 10 according to the exemplary embodiment is a method of treatment using a stent delivery system 10 configured to deliver the stent 200, which is located between the distal portion of the inner tube 20 having the guide wire lumen 21 formed therein and the distal portion of the outer tube 30 arranged to be relatively movable with respect to the inner tube 20, to a desired position in the living body lumen, and includes (i) a process of indwelling the stent 200 in the first lesion area X1 of the first blood vessel V1, (ii) a process of removing the inner tube 20 from the outer tube 30, (iii) a process of inserting the medical device 500 into the living body lumen via the lumen 31 of the outer tube 30, and (iv) a process of carrying out a procedure in the living body lumen using the medical device 500.

Furthermore, the medical device 500 includes at least one of a balloon catheter, a self-expandable stent delivery system, a balloon-expandable stent delivery system, an angiographic catheter, an ultrasonic catheter, an atherectomy catheter, an endoscopic catheter, a drug delivery catheter, and a microcatheter.

Moreover, the process of inserting the medical device 500 into the living body lumen via the lumen 31 of the outer tube 30 includes a process of inserting the medical device 500 into a second blood vessel V2 located nearer a distal end side than the first blood vessel V1.

Additionally, the process of carrying out a procedure in the living body lumen using the medical device 500 includes a process of carrying out a procedure on the first lesion X1 using the medical device 500, and/or a process of carrying out a procedure on the second lesion X2 formed in the second blood vessel V2 using the medical device 500.

Furthermore, the first blood vessel V1 is the superficial femoral artery, and the second blood vessel V2 is the popliteal artery.

As described above, according to the stent delivery system 10 in the exemplary embodiment, since, with the stent 200 released, the inner tube 20 and the distal member 23 are configured to be removable from the outer tube 30 via the lumen 31 of the outer tube 30, the inner tube 20 and the distal member 23 can be removed from the outer tube 30 after the stent 200 is indwelled in the living body lumen. With this, the balloon catheter 500 can be guided into the living body lumen via the lumen 31 of the outer tube 30. In this way, utilizing the outer tube 30 as a guiding catheter for the balloon catheter 500 enables reducing the cost of a guiding catheter and thus contributing to medical economy. Furthermore, since the step of inserting a guiding catheter can be removed, a prompt procedure can be performed, so that a less-invasive procedure with the burden on the patient reduced can be implemented.

Moreover, there is further included a connector portion 120 arranged at the proximal end side of the outer tube 30 and arranged to be movable together with the outer tube 30. The connector portion 120 includes a main tube 121, which communicates with the lumen 31 of the outer tube 30 and through which the inner tube 20 is movably inserted, a branch tube 122, which branches from the main tube 121 and which has a port 122a formed therein that communicates with the lumen 31 of the outer tube 30 via the main tube 121, and a valve body, which is arranged at the branch tube 122 and which allows a fluid to flow from the port 122a to the lumen 31 of the outer tube 30 but, on the other hand, prevents a fluid from flowing from the lumen 31 of the outer tube 30 to the port 122a. Therefore, while a contrast agent or the like can be injected from the port 122a of the branch tube 122 into the lumen 31 of the outer tube 30, a blood inflowing from the distal end of the outer tube 30 can be prevented from flowing out of the branch tube 122 via the port 122a. With this, a user's operability can be improved.

In addition, there is further included a marker 32 having radiopacity, which is arranged at the distal portion of the outer tube 30. With this, the marker 32 enables clearly confirming the position of the distal portion of the outer tube 30 on a radiographic image. Thus, positioning of the balloon catheter 500 in the living body lumen can be accurately and promptly implemented.

While a self-expandable stent delivery system according to the disclosure herein has been described above through an exemplary embodiment thereof, the invention is not limited to only the configuration described in the embodiment, but can be changed as appropriate based on the description set forth in the claims.

For example, while, as a treatment site, the lower extremity of a patient is described by way of example, the treatment site to which a self-expandable stent delivery system according to the disclosure is applicable is not limited to the lower extremity, but can be applied to other sites in the living body lumen, such as upper extremities and the heart.

Furthermore, in treatment, a medical device that is inserted through the outer tube when it is used as a guiding catheter is not limited to a balloon catheter, but can include, for example, according to appropriate uses, a self-expandable stent delivery system, a balloon-expandable stent delivery system, an angiographic catheter, an ultrasonic catheter, an atherectomy catheter, an endoscopic catheter, a drug delivery catheter, and a microcatheter.

Moreover, in the self-expandable stent delivery system according to the disclosure, as long as the inner tube and the distal member can be configured to be removable from the outer tube via the lumen of the outer tube, structures of various portions, locations of various members, and others thereof can be changed as appropriate.

Additionally, the omission of any additional member described with reference to the drawings or the use of another additional member can be made as appropriate.

The detailed description above describes features and aspects of an embodiment of a self-expandable stent delivery system and a method for using the same. The invention is not limited, however, to the precise embodiment and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A self-expandable stent delivery system comprising:
an inner tube having a guide wire lumen formed therein, and through which a guide wire is insertable;
an outer tube including a lumen, through which the inner tube is insertable, and arranged to be relatively movable with respect to the inner tube;
a distal member fixed to a distal end of the inner tube; and
a self-expandable stent arranged between a distal portion of the inner tube and a distal portion of the outer tube and configured to expand and deform after being released from between the inner tube and the outer tube along with movement of the outer tube,
wherein, after release of the self-expandable stent, the inner tube and the distal member are configured to be removable from the outer tube via the lumen of the outer tube.

2. The self-expandable stent delivery system according to claim 1, further comprising a connector portion arranged at a proximal end side of the outer tube and arranged to be movable together with the outer tube relative to the inner tube,
wherein the connector portion includes:
a main tube which communicates with the lumen of the outer tube and through which the inner tube is movably inserted;
a branch tube which branches from the main tube and which has a port formed therein communicating with the lumen of the outer tube via the main tube; and
a valve body configured to allow a fluid to flow from the port to the lumen of the outer tube and further configured to prevent a fluid from flowing from the lumen of the outer tube to the port.

3. The self-expandable stent delivery system according to claim 2, further comprising a proximal shaft which covers the inner tube at a proximal end side of the connector portion.

4. The self-expandable stent delivery system according to claim 3, further comprising an on-off valve arranged to surround an outer periphery of the proximal shaft, wherein when the on-off valve is in an open state, the connector portion is relatively movable with respect to the proximal shaft and when the on-off valve is in a closed state, movement of the connector portion relative to the proximal shaft is restricted.

5. The self-expandable stent delivery system according to claim 4, wherein the on-off valve comprises a cap portion including a male thread portion formed on an outer surface of a proximal portion of the main tube and a female thread portion threadedly engageable with the male thread portion.

6. The self-expandable stent delivery system according to claim 1, further comprising a marker having radiopacity, which is arranged at the distal portion of the outer tube.

7. The self-expandable stent delivery system according to claim 1, further comprising a marker having radiopacity, which is fixed to an outer surface of the inner tube.

8. The self-expandable stent delivery system according to claim 7, wherein frictional forces generated due to the marker prevent the outer tube from moving with respect to the inner tube.

9. The self-expandable stent delivery system according to claim 1, wherein the distal portion of the inner tube is configured to protrude beyond the distal portion of the outer tube, a gap portion being provided between the distal portion of the inner tube and the distal portion of the outer tube to accommodate the stent.

10. The self-expandable stent delivery system according to claim 9, wherein a stent stopper is disposed near a proximal end of the stent when the stent is accommodated in the gap portion.
